(19) United States
(12) Patent Application Publication
(10) Pub. No.: US 2008/0236575 A1
(43) Pub. Date: Oct. 2, 2008

(54) INTUBATION DEVICE WITH VIDEO, STYLET STEERING, PREP AND STORAGE SYSTEM

(76) Inventor: Robert Michael Chuda, Yonkers, NY (US)

Correspondence Address: DONALD W. MEEKER, 924 EAST OCEAN FRONT, # E, NEWPORT BEACH, CA, 92661, US

(21) Appl. No.: 12/058,492

(22) Filed: Mar. 28, 2008

Related U.S. Application Data
(60) Provisional application No. 60/920,539, filed on Mar. 29, 2007.

(51) Int. Cl. A61M 16/00; A61B 1/267

(52) U.S. Cl. 128/200.26; 600/120

(57) ABSTRACT

A handle with a video display is attached to a flexible stylet containing firming and bending tendons. Trigger controlled anatomic shaping provides steerage to the stylet which is inserted into an endotracheal tube or other tube for insertion into body orifices. The stylet and mounted endotracheal tube are guided by a video image from a camera and light on the distal end of the stylet. A storage container and sterilization and prep kit enable the device to be immediately available for reuse when needed.

### CROSS-REFERENCE TO RELATED APPLICATIONS

The present utility patent application claims the benefit of provisional application No. 60/920,539, filed Mar. 29, 2007

### STATEMENT REGARDING FEDERALLY SPONSORED RESEARCH OR DEVELOPMENT

Not Applicable.

### THE NAMES OF THE PARTIES TO A JOINT RESEARCH OR DEVELOPMENT

Not Applicable.

### BACKGROUND OF THE INVENTION

1. Field of the Invention

The present invention relates to medical devices useful to aid the intubation, or placing of tubes. in the accessible body cavities of patients. More particularly, the preferred embodiment of the present invention is a device to aid in the placement of endotracheal tubes (breathing tubes) such that ventilation and oxygenation of the lungs can be accomplished. Even more particularly, the present invention is designed to aid intubation procedures in the setting of anatomic or other difficult conditions which might prevent intubation from being successfully performed using routine equipment and methods. Additionally, the present invention relates to the cleaning, disinfecting, lubricating and defogging of these devices and storing them such that they can be readily utilized in emergency as well as routine intubations.

2. Description of Related Art Including Information Disclosed Under 37 CFR 1.97 and 1.98

Currently, the majority of endotracheal intubation procedures are effected by the use of a rigid laryngoscope inserted through the mouth. The endotracheal intubation procedure involves passing a tube via the mouth or nasal passages through the pharynx (area at the back of the mouth and nose), past the tongue and epiglottis, and through the vocal cords (glottis) into the trachea and the endotracheal intubation device removed. This tube is then used to maintain the airway and support ventilation of the lungs, a critical life support function. Using a straight or curved blade with a light along its length, the tongue and epiglottis are lifted out of the way allowing direct visualization of the vocal cords such that an endotracheal tube can be advanced into the trachea.

In cases where direct visualization of the opening to the larynx is not possible, due to anatomic variation, trauma, or pathologic process, the procedure may be difficult causing injury or even failure to be successfully performed. Conditions such as increased hypopharyngeal tissue as found in morbid obesity. short muscular neck, cephalad location of the larynx, prominent teeth, mandibular shortening, inability to open the mouth sufficiently, inability to position and manipulate the head and neck (such as cervical spine fixation or injury), tumors or masses of the oropharynx or larynx can all prevent direct laryngoscopy necessary for intubation with a rigid laryngoscope.

If a difficult intubation for one of the foregoing reasons is anticipated in advance (elective or non-emergent situation), certain methods and devices would then be used to secure the airway. Intubation can be accomplished over a flexible fiber optic bronchoscope or fiber optic laryngoscope. These devices are expensive and very delicate, and require considerable expertise to use effectively. In the non-emergent case, frequently an expert with this equipment will be notified in advance, since neither the expert or the fiber optic scope are readily available on short notice. Also, the construction and flexibility of these devices makes them awkward, even in the expert's hands, especially for orotracheal intubation, as the typical flexing pattern of these types of scopes at times cannot navigate the angles necessary for orotracheal intubation and can only be used successfully via the nasal route. These fiber optic devices also suffer from degradation in visual transmission quality due to bending or breaking of the individual fiber optic bundles with repeated use. In between procedures, these devices must be formally processed for cleaning and sterilization, as they often come into direct contact with mucus membranes and have channels or conduits within the scope which become contaminated by trapping secretions.

This need for high level decontamination or sterilization processing typically removes the device from availability for use for a period of time, usually over an hour and perhaps even several hours or the entire day. Since these devices are delicate and expensive, most hospitals own a limited number of them, and down time during reprocessing can result in lack of availability of necessary equipment in emergency situations. After sterilization, they are typically stored with other emergency and difficult airway management devices in a central location or on a moveable ‘airway cart’ found in many operating rooms and other anesthetizing locations. Fiber optic intubation in the conscious, spontaneously breathing patient is considered the gold standard of securing the airway in patients that cannot be intubated via direct laryngoscopy, but is a difficult and time consuming process requiring considerable training and expertise.

However. this technique of conscious fiber optic intubation can rarely be used in the setting of unanticipated difficult intubation, such as when direct laryngoscopy fails during induction of general endotracheal anesthesia and the patient has received paralyzing drugs necessary as surgical muscle relaxants and the anesthesiologist must breath for the patient immediately. This crisis situation, known as CICV: Can't Intubate, Can't Ventilate’ can have a fatal outcome. Numerous devices, such as specialized rigid laryngoscopes, oral and nasal pharyngeal airways, laryngeal masks, or esophageal airways may be inserted blindly (without the aid of a laryngoscope) to assist delivery of oxygen to the patient.

Most of these devices do not incorporate a video display, but transmit the distal image to an optical eyepiece, which requires positioning the intubator close to the patents mouth where potential exposure to vomitus or other secretions may occur. A separate video camera can often be attached to these devices. The use of a video screen rather than an optical eyepiece is advantageous to allow simultaneous viewing of both the internal and external condition of the patient. These separate light source and video cameras and displays also require separate focusing, control boxes, AC power cords and convenient AC outlets for use. This prohibits use in field conditions which might be found by EMT or ambulance responders.

However, none of these devices, by themselves, enter the trachea. Therefore, such devices may not provide a permanent solution for intubation of the trachea which is necessary for ventilation of the patient. the prevention of aspiration, respiratory therapy or certain surgical procedures. These devices all have fixed curves and endotracheal tube guide paths which may not be suitable for all patients.

Devices designed to enter the trachea are disclosed in the prior art, but do not adequately solve all the problems.

George, U.S. Pat. No. 4,742,819 discloses a semi-malleable rigid stylet placed inside the endotracheal tube, with an external video screen. An alternative embodiment described utilizes a lever to manipulate the distal end of the device, but its dependence on the pre-formed curve of the remaining stylet may be inadequate for all anatomic variations encountered. Also fiber optic bundles may break with repeated use, degrading the video signal.

Greene, U.S. Pat. No. 5,327,881 discloses a rigid fiber optic intubating stylet with distal acute angle tip angulation. This device suffers from fiber optic bundle degradation and well as non-anatomic shaping of the stylet portion. Having an eyepiece only, it lacks video display, forcing the user to place their eye in proximity of exposure to patient expectorations.

Flarn, U.S. Pat. No. 5,607,386 discloses an apparatus to guide fiber optic bronchoscopes with mounted endotracheal tubes into the trachea, but suffers from the use of fiber optics, and the bronchoscope with conduits must be taken out of service for time-consuming cleaning and sterilization. The device is large and cumbersome in use as depicted in the drawings.

Raybum et al, U.S. Pat. No. 5,733,242 discloses an intubation system which can be located in the trachea and an endotracheal tube advanced over it into position. This device lacks a video display and suffers from fiber optics and a pre-bent shape which is not adjustable during use, except for a short tip portion similar to George, above, with a lens that needs to be treated to prevent fogging. The presence of conduits require formal cleaning and sterilization.

Bashour, U.S. Pat. Nos. 5,803,898 and 6,432,042 discloses an ‘endoscopic stylet’ which is semi rigid, and requires the user to ‘anticipate the airway contours’ in that the device lever only steers the distal tip. This device suffers not only from fiber optics and the lack of a video display, but also must be pre-formed into what might be the correct shape for the individual patient, and must be withdrawn, re-shaped and reinserted. It has no ergonomic handle, and the stylet with the mounted endotracheal tube must be awkwardly held directly.

Sanders et al. U.S. Pat. No. 5,913,816 discloses an intubation device and method which describes in general terms the theory of every endoscopic intubation device. The disclosed device needs separate light and camera sources and monitors, as well as utilizing conduits which dictate formal cleaning and sterilization procedures. This requirement is partially circumvented by requiring the use of disposable components, which complicate the use of this device.

Nakaichi et al, U.S. Pat. Nos. 6,004,263, 6,319,195 and 6,432,043 discloses an endoscope for intubating having a bendable stylet element, but it only deflects in one direction, and may not be suitable for all patients. It also suffers from the utilization of fiber optics.

Schwartrz et al, U.S. Pat. No. 6,539,942 discloses an endotracheal intubation device bendable during use into an L-shaped configuration at the distal end to promote visualization of the vocal cords. Since the anatomic path of a properly placed endotracheal tube has never been described as ‘L-shaped’, it is not clear just exactly how this device could facilitate intubation. This device must also be used with a separate ‘scope’ device.

Chhibber et al, U.S. Pat. No. 6,832,986 discloses an endoscopic intubation system which is not bendable or adjustable, and designed specifically for newborn babies and suffers from fiber optic bundle effects.

Hill, U.S. Pat. No. 6,929,600 discloses a semi-malleable rigid stylet placed inside the endotracheal tube. with an external video screen. But the stylet is not adjustable during use, and must be withdrawn from the patient, readjusted, and the procedure reattempted if the initial user adjustment is not suitable for that particular patient. This device does not describe a handle. and the stylet with the mounted endotracheal tube must be awkwardly held directly. Withdrawing this semi-rigid device along the length of the endotracheal tube after accomplishing intubation can also be difficult, as curve angles typically differ along the length of the tube, sometimes dislodging the endotracheal tube from its proper position. Conduits require formal cleaning and sterilization.

The present invention is designed to overcome the disadvantages of the prior art, while combining several desirable objectives in achieving successful endotracheal intubation as well as sterilization, maintenance and storage of the elements of the system.

### BRIEF SUMMARY OF THE INVENTION

It is a primary object of the present invention to provide a device which incorporates a locking ball joint adjustment relationship between the handle/video monitor and the working stylet/endotracheal assembly, which provides ease of use for any intubator even in awkward patient positions (e.g. car wreck, during neurosurgery, etc.) for an easily guided naturally bending intubation tube with conforms to the actual shapes of the patient passageways and utilizes a camera system mounted on the device with a viewing screen on the handle to observe the path of the intubation tube simultaneously with the observation of the external condition of the patient, and an ergonomically shaped handle which optimizes manipulation of the invention.

It is a corollary object of the present invention to provide complete prep (cleaning, disinfecting, defogging, lubricating) capabilities and provision for safe immediately accessible operating room storage.

It is an objective of the present invention is to provide a device for intubation which is easy to use and readily available without the delay or expertise inherent in the use of fiber optic bronchoscopes or laryngoscopes.

It is a further objective to provide an intubation device which can be used orally or nasally.

It is a further objective to provide an intubation device with a self contained power source.

It is a further objective of the present invention to visually facilitate intubation using a tip located camera that does not utilize fiber optics, allowing observation of the internal and external condition of the patient.

It is a further objective to manually steer placement of the endotracheal tube by changing stylet shape to conform to the range of anatomical shapes necessary to navigate the entrances to the trachea in a variety of patients' individual situations, and then allow flaccid withdrawal of the stylet without dislodging the endotracheal tube.

It is a further objective to allow intubation of patients whose anatomy or clinical condition precludes direct laryngoscopy or other routine methods of intubation, and to accomplish intubation in situations where direct laryngoscopy is impossible or contraindicated.

A yet further object is to combine and improve upon aspects of prior endoscopic stylet video intubation systems to create a unique instrument incorporating the strengths of these systems and eliminating the weaknesses of the aforementioned systems.

It is a further objective to provide an intubation device in which the stylet portion is sealed, without conduits, and does not come into contact with patient mucus membranes, which uses a system to clean, disinfect, lubricate and defog the intubation device to allow rapid reuse for the next patient.

It is a further objective to provide a container to store and protect the intubation device in the location of proposed use, such that the device is immediately available.

In brief, the preferred embodiment of the present invention relates to a system for inserting an endotracheal tube within a patient. More particularly the present invention relates to a steerable video intubation device, comprising an ergonomic handle with video display and trigger lever adjustor connected by means of a ball and socket type joint to a flexible stylet containing steering and stiffening tendons, and a distal tip lens-camera-light source. The device is cleaned, disinfected, lubricated and defogged with cloth(s) wrapped in foil packaging formulated for these purposes and compatible with materials used in its manufacture. An endotracheal tube is then slid over the stylet and secured to the endotracheal tube stop. The intubation device with mounted endotracheal tube uses its anatomic shaping, mechanical steering and video capabilities to allow insertion of the tube into the patients' trachea, at which time the device is withdrawn, leaving the endotracheal tube in the trachea. The storage container provides protection and handy availability of the prepped device in various urgent and routine clinical situations.

The intubation device of the present invention provides ease of use for any intubator even in awkward patient positions (e.g. car wreck, during neurosurgery, etc.) for an easily guided naturally bending intubation tube with conforms to the actual shapes of the patient passageways and utilizes a camera system mounted on the device with a viewing screen on the handle to observe the path of the intubation tube simultaneously with the observation of the external condition of the patient, and an ergonomically shaped handle which optimizes manipulation of the invention.

This and other uses of the invention will be become more obvious through the consideration of the drawings of preferred embodiments. Any accessible body cavity such as lungs, stomach, vagina, anus and rectum can be intubated or examined with different embodiments of the device.

### BRIEF DESCRIPTION OF THE SEVERAL VIEWS OF THE DRAWINGS

These and other details of the present invention will be described in connection with the accompanying drawings, which are furnished only by way of illustration and not in limitation of the invention, and in which drawings:

FIG. 1 is a side elevational view of an intubation device in accordance with the present invention;

FIG. 2A is a side elevational view in partial section of the intubation device of FIG. 1, demonstrating the lever controlled tendon mechanism to deflect, bend and firm the stylet;

FIG. 2B is an enlarged cross sectional view of the stylet portion of the intubation device taken at 2′-2′ of FIG. 2A;

FIG. 3A is a front elevational view of a single foil pouch of the present invention;

FIG. 3B is a front elevational view of a double foil pouch of the present invention;

FIG. 3C is a front elevational view of a quadruple foil pouch of the present invention;

FIG. 3D is a cross sectional view of a foil pouch with multiple internal layers taken at 3′-3′ of FIG. 3C;

FIG. 4A is a front elevational view of a storage container in accordance with the present invention;

FIG. 4B is a side elevational view of the storage container of FIG. 4A;

FIG. 4C is an elevational view of a impermeable sheath to isolate the stylet portion of the present invention.

### DETAILED DESCRIPTION OF THE INVENTION

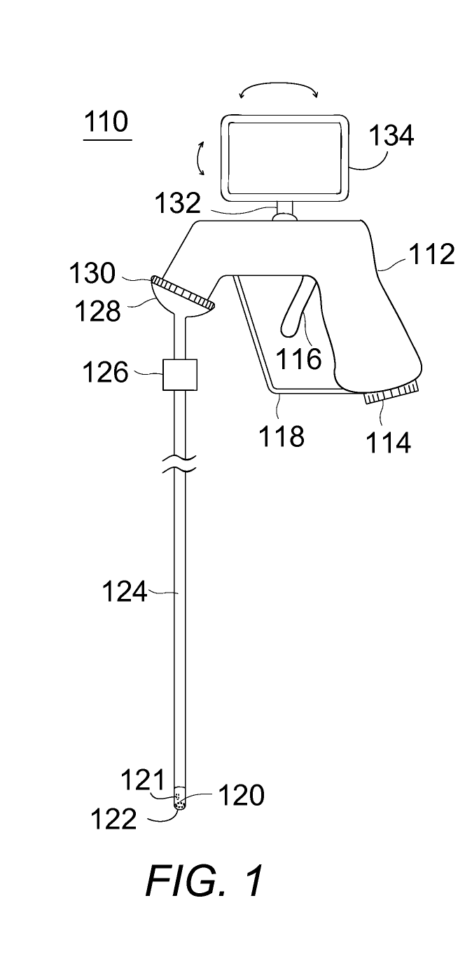

In FIGS. 1-4, a passageway conforming intubation system comprising a self-contained guided intubation device 110 for intubating passageways of patients, a prep kit 300 providing cleaning, disinfecting, defogging, and lubricating capabilities for the device, and a safe immediately accessible operating room storage container 400 for the system.

Referring to FIG. 1, the intubation device 110 comprises an ergonomically shaped handle 112 connected to a stylet 124 via a ball and socket type joint 128 with a locking screw down collar 130. The handle 112 is ergonomically formed to fit the users hand and may be pistol shaped (depicted in FIG. 1) or fusiform, or based on a mold taken of the human hand. The handle 112 is preferably made from medical grade material and may be constructed from metal, such as aluminum, or high quality plastic or other polymer.

The stylet 124 is made from a medical grade bendable, compressible polymer. The distal end of the handle 112 bends or angles down (depicted) to provide user ergonomics during the intubation procedure, as the typical entrance to a patients' mouth is vertically oriented when the patient is supine, which is the usual position for an intubation procedure. The ball and socket type joint 128 allows further adjustment of the handle-to-stylet angle to accommodate varying patient anatomy and positioning. The ball and socket joint and locking collar are conventional and not further described.

A lever 116 extends from the handle 112, which preferably acts as a time limited on-off switch activating the light source and video display. This lever also causes bending and extension of the stylet 124. The stylet 124 bends along its entire length approximating curves of progressively smaller radiuses. The range of the induced bend from the neutral position is preferably approximately 160 degrees to minus 30 degrees. The lever is surrounded by a guard 118. Battery access 114 is provided at the handle bottom for accessing the rechargeable battery 214 in the handle.

A video display 134 preferably of the LED type, is attached to the handle via a rotating and tilting strut which inserts into a plug 132 at the top of the handle. The plug 132 also allows connection of the handle to an external video monitor and/or recorder (not depicted). The stylet 124 is sealed, without external openings to conduits. This distal tip of the stylet 122 is smooth with rounded edges to prevent laryngeal injury during the intubation procedure.

The distal tip of the stylet 122 contains a video camera with prefocused lens 120 providing suitable focal length and depth of field for the intubation procedure, as well as a warming element 121 to heat the tip 122 to normal body temperature as an aid to prevent lens fogging. The camera light, lens, and warming element are conventional and not further described.

A stop 126 or other method to secure an endotracheal tube mounted over the stylet is provided. This element is adjustable along the length of the stylet 124 and is secured to the stylet shaft either frictionally, by clamp. or cam lock. or other conventional means.

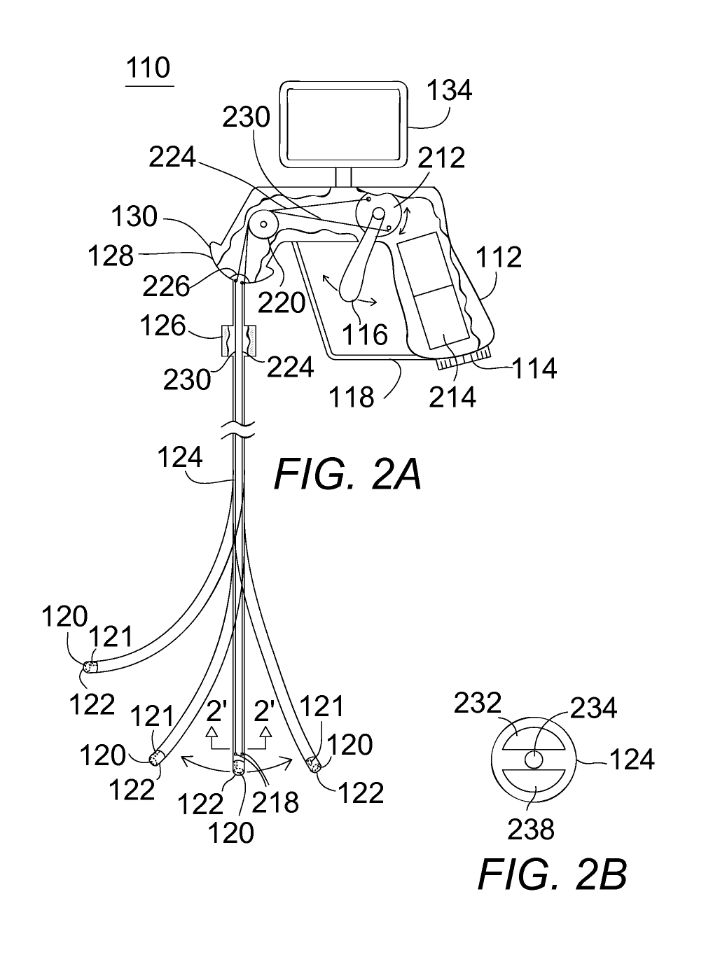

Referring to FIG. 2A, the intubation device inner mechanism is shown in partial cross-sectional view. The battery 214 is contained within the handle. Demonstrated is the method by which the lever 116 imparts bending and stiffening to the flexible stylet 124. Manipulation of the lever 116 rotates a spool 212 which may be round (depicted), clam, or cam shaped. At least one tendon, but preferably two 224 and 230, are advanced into or retracted from, the stylet, affecting its shape and stiffness. Depicted are two tendons, where one tendon 224 acts to cause downward or posterior deflection, while a second tendon 230 controls upward or anterior deflection of the stylet. The course of these tendons from the lengthening/shortening spool 212 passes over a proximal, large guide 220, rotates 90 degrees, and then a over a distal, small guide 226 for proper orientation in the stylet shaft. The tendons are anchored 218 at the distal end of the tendon race/guide way. The stylet 124 bends smoothly along its length approximating curves of progressively smaller radiuses. The range of the induced bend from the neutral position is approximately 160 degrees to minus 30 degrees. FIG. 2B is an enlarged cross section of the stylet shaft 124 taken at 2′-2 depicting the tendon race/guide channels for the anterior 230 and posterior 224 steering and stiffening tendons. A central channel for wiring 234 is depicted, but the wiring can alternately be molded into the substance of the stylet shaft at time of manufacture.

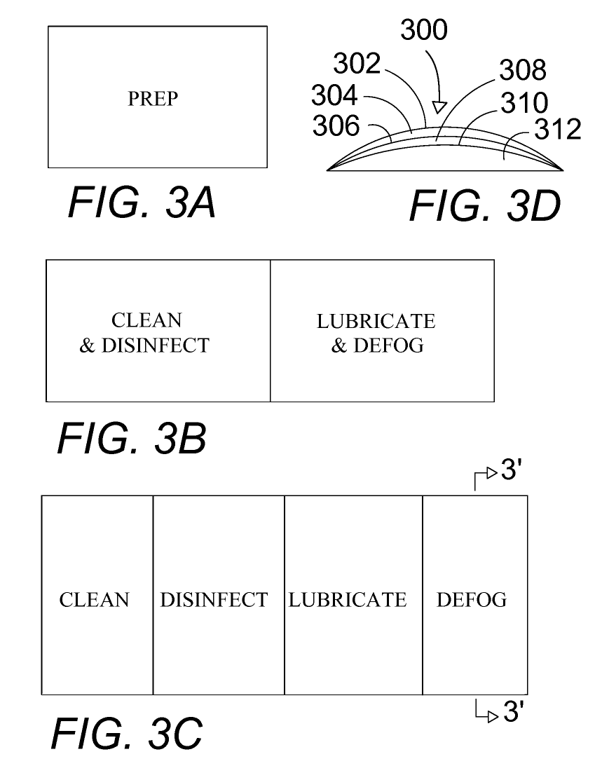

FIG. 3A is a front view of a single foil pouch. FIG. 3B is a front view of a double foil pouch, and FIG. 3C is a front view of a quadruple foil pouch, labeled to indicate functional contents. These pouches contain either singly or in combination cloths and chemicals which function to clean, disinfect, lubricate, and defog the intubation device. The cloth is preferably soft, lint-free, disposable, and absorbent; suitable for cleaning or applying chemicals, as the intended use may be. The chemicals may be enzymatic cleaners, detergents, peroxides, peracetic acid, glutaraldehydes, alcohols, chlorinated bleaches, silicones such as dimethylpolysiloxane, defoggers, or substances which when combined release heat. FIG. 3D is a cross section view of either pouch taken at 3′-3′ with multiple internal separations. The separating barriers may be designed to burst upon squeezing of the pouch to combine chemicals or apply chemicals to cloths at time of use.

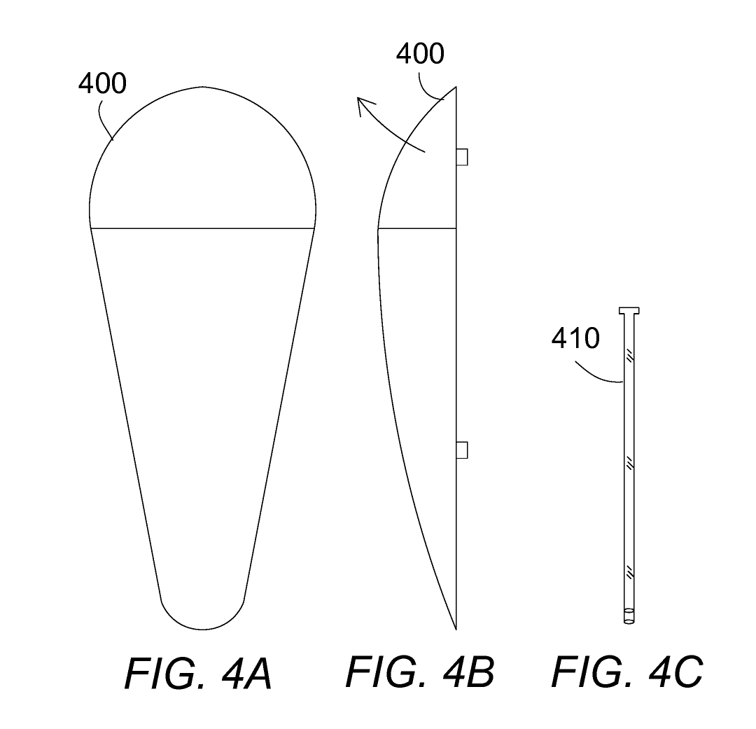

FIG. 4A depicts a front view of a storage container for the intubation device: FIG. 4B depicts a side view. The storage container stores the prepped intubation device and several prep packages. The shape of the storage container provides snag free ergonomics when mounted in the operating room.

### Operation—Preferred Embodiment

In operation, the user removes the prepared intubation device from the storage container FIG. 4B. It is intended that the intubation device be stored clean and chemically treated for the next use, to allow immediate application to any intubation procedure. The immediate user may re-prep the device, especially if the applied lubricant and defogging chemicals have dried or evaporated due to prolonged storage. Lubrication, preferably with a silicone-based compound, is essential to allow movement of the stylet 124 relative to the endotracheal tube to allow bending of the endotracheal tube-stylet portion and easy withdrawal of the stylet 124 at the conclusion of the intubation procedure. Defogging may be achieved by application of a chemical defogger and/or heating tip 121 to heat the end of the stylet 124 and camera to body temperature by insertion into a foil packet FIG. 3A-3D containing just combined mixture of chemicals which release heat, such as conventional hand warming packets which skiers use to warm their hands inside their gloves. FIGS. 3B and 3D depict such a packet, where the packet labeled “LUBRICATE & DEFOG” contains a cloth saturated with a lubricating silicone based chemical in lumen 312 and the heating chemicals separated from reacting in storage in lumens 308 and 304. When squeezed, the separating layer 306 bursts and allows combination of the heating chemical mixture, which imparts warming to the stylet placed into the lumen containing the cloth and lubricating chemical(s) Alternatively, the video camera lens 120 may be electrically warmed by a heating element powered by the handle power source 214.

A conventional endotracheal tube is mounted on the stylet 124 and frictionally secured to the endotracheal tube stop 126 via its universal connector. The endotracheal tube is positioned on the stylet 124 such that the tip 122 is almost flush, but not protruding from the endotracheal tube. The articulating joint 128 allows adjustment of the angle between the handle 112 and the stylet 124 to accommodate use of the device for individual users (right vs. left handed intubators), unusual patient anatomy, and different patient positioning situations, which may indicate different angles of the relation of the handle to the stylet for comfortable and efficient use. The trigger lever 116 activates the light source and camera 120, and video display 134 for a predetermined period of time. The video display 134 may also indicate battery charge status and/or elapsed time from activation. The plug 132 on top of the handle 112 connects the video display 134 to the device. Alternatively the plug 132 can be used to connect with an external video monitor and/or recorder (not depicted) if the user desires a larger screen display and/or recording of the intubation procedure.

During use, the trigger lever 116 adjusts the stylet 124 by jamming or tensioning the tendons 224 and 230, shaping the stylet 124 into an anatomic curve. One tendon 224 acts to cause downward or posterior deflection, while a second tendon 230 controls upward or anterior deflection of the stylet. Stiffness is imparted by the tensioning and jamming of the tendons in their raceways 232 and 238 (FIG. 2B) resisted by the preformed shape of a conventional endotracheal tube. The stylet 124 bends smoothly along its length approximating curves of progressively smaller radiuses. The range of the induced bend from the neutral position is approximately 160 degrees up or anteriorly, to minus 30 degrees down or posteriorly. Thus the stylet can assume a range of anatomic shapes necessary to navigate the airway passages. The amount of force applied to the lever controls the amount of bend and inducement of curves of progressively smaller radius along the stylet. The device with endotracheal tube mounted thereon is introduced into the patients' hypopharynx, orally or nasally. The lens-camera 120 focal length and depth of field are preset to display anatomic structures encountered during the intubation procedure. In the unconscious patient unable to maintain an open airway, a conventional direct laryngoscope may be used to lift the tongue and epiglottis out of the way. Using overall manipulation of the stylet and steering mechanism, the device with mounted endotracheal tube is advanced past the epiglottis until the vocal cords are identified video graphically.

The device with mounted endotracheal tube is advanced through the vocal cords, thus positioning the endotracheal tube in the trachea, also videographically identified by its typical appearance. The endotracheal tube is then disconnected while the device is withdrawn, leaving the endotracheal tube in the trachea. The device is then cleaned, disinfected, lubricated and defogged with the cloth(s) of the cleaning and prep system contained in the dedicated foil pouch(es) FIG. 3A-C. The cloth is preferably soft, lint-free, disposable, and absorbent; suitable for cleaning or applying chemicals, as the intended use may be. Since the intubation device does not enter tissues normally considered to be sterile, and is not intended to come into contact with any mucus membranes of the patients' airway or respiratory system, formal sterilization is not required. The sealed construction of the stylet without conduits does not trap patient secretions which may contaminate the stylet during intubation procedures. Adequate cleaning is accomplished with enzymatic cleaners, detergents. or other suitable agents approved by the FDA for this purpose. After cleaning, disinfection is accomplished by exposure to chemicals such as peroxides, peracetic acid, glutaraldehydes, alcohols. chlorinated bleaches. or other topical disinfectants approved by the FDA for this purpose. Lubrication and defogging is provided by substances which may contain silicones such as dimethylpolysiloxane, or other lubricating chemicals, and defoggers, or substances which when combined release heat The intubation device is then stored in its container, providing protection and handy availability of the prepped device and foil wrapped prepping cloths in various urgent and routine clinical situations. The storage container FIG. 4A-4B is mounted vertically in the operating room, on the wall, anesthesia machine, or supply cart. The smooth shaping and rounded edges of the storage container provide a non-snagging ergonomic location in the crowded setting of a modern operating room, allowing instant availability for any intubating procedure.

DESCRIPTION—Additional Embodiment Pediatric and neonatal endotracheal tubes are much smaller and shorter than adult endotracheal tubes, and require a smaller sizing of the stylet 124 portion of the intubation device.

An intubation device to aid the placement of endobronchial tubes for separate lung ventilation and one lung anesthesia requires the stylet 124 portion to be longer.

An intubation device to aid other body cavity endoscopy. intubation, or surgery such as ENT diagnostic. gastroscopic, or proctoscopic model, can be utilized with protection of the device by a impermeable sheath or condom. FIG. 4C depicts a sheath or condom for protecting alternate embodiments of the intubation device during procedures when the stylet may come into contact with sterile body cavities. The length of the stylet 124 portion would vary accordingly for these specialized uses.

Another alternative embodiment eliminates the ball and socket joint 128, or utilizes other conventional means of adjusting handle to stylet angle.

### Operation

Additional Embodiment The operation of additional embodiments described above is essentially the same as that described for the preferred embodiment used for endotracheal intubation. Other body cavities would be approached by the appropriate external orifice and the protective sheath 410 of FIG. 4C used with the intubation device if sterile tissues are expected to be encountered.

### CONCLUSIONS, RAMIFICATIONS, AND SCOPE

The present invention overcomes all of the inadequacies of the intubation devices seen in the prior art. It can be used in the conscious or unconscious (anesthetized) patient, orally or nasally. It is adjustable during use to conform to the individual anatomy of the patient, the anatomic shaping by trigger lever promoting endotracheal tube placement without head, neck or jaw manipulation. The prefocused integral video display eliminates the need to place the intubators' head near the patients mouth with exposure to vomitus or other secretions. There are no fiber optical light transmission bundles to break with image degradation. The battery powered embodiment does not require power cords or AC availability for use. It is designed not to come into contact with mucus membranes and its one piece sealed construction without conduits is quickly and easily cleaned, disinfected, defogged and lubricated with the compatible prep system for subsequent use. Simple construction with available technology provides easy affordability and the protective case provides secure storage and immediate availability in emergency as well as routine situations. It is meant to be used for all intubations to allow familiarity with use when difficult situations arise unexpectedly.

The intubation device of the present invention provides a tool useful for intubation procedures where usual methods can fail. This device can not only be used for endotracheal intubation procedures, but different embodiments can be used for specialized intubations, or assessment and surgery of any accessible body cavity.

It is understood that the preceding description is given merely by way of illustration and not in limitation of the invention and that various modifications may be made thereto without departing from the spirit of then invention as claimed.

What is claimed is:

1. A self-contained guided intubation device for intubating passageways of patients, the device comprising in combination:
a handle structured in an ergonomic shape to fit a hand of a user for comfortable use;
an adjustment lever protruding from the handle in communication with the fingers of a user grasping the handle;
a flexible, adjustable stylet attached to the handle and extending vertically downward therefrom, the stylet releasably holding a tube for insertion into a passageway of a patient, the stylet comprising flexing, extending and stiffening tendons within the stylet controlled by the adjustment lever so that the stylet bends along its entire length approximating curves of progressively smaller radiuses to cause the stylet to conform to the anatomically accurate actual shape of the passageway of the patient so that the stylet travels an anatomically accurate path into the passageway of the patient;
a lens, camera and light source located at a distal tip of the stylet electrically connected to transmit a real time video image of the view in front of the stylet as the stylet is inserted into the passageway of the patient;
a power source and electrical connectors connected to the camera and the light source;
a video display attached to the handle portion to receive the real time video image from the camera, the video display visible to the user holding the handle portion, the user simultaneously having visual communication with the exterior of the patient at the location of the insertion of the stylet so that the system provides a steerable video monitored intubation device with an insertion tube releasably mounted on a stylet anatomically accurately shaped in real time using mechanical steering and video capabilities to allow insertion of the insertion tube into the passageway to a desired point and withdrawal of the stylet leaving the insertion tube in place in the passageway of the patient, thereby providing a self-contained guided intubation device for intubating passageways of patients.

2. The intubation device of claim 1 further comprising an adjustable locking ball joint a between the handle and the stylet.

3. The intubation device of claim 1 wherein the adjustment lever is connected to the handle by a pivot wheel having a first cable attached from a first side of the pivot wheel to steering tendons in the flexible stylet and a second cable attached from a second side of the pivot wheel to stiffening tendons in the flexible stylet so that the stylet is adjustable to conform to the shape of the passageway of the patient by controlling the adjustment lever.

4. The intubation device of claim 1, wherein the flexible condition of the stylet is variably stiffened and curved into a range of anatomic shapes suitable to guide the endotracheal tube from outside a patient, through the nasopharynx or oropharynx and through the vocal cords into the trachea.

5. The intubation device of claim 1 wherein the video display displays real time visual landmarks of airway anatomy that are electrically transmitted by the tip camera to the video display.

6. The intubation device of claim 1 wherein the power source is contained in the handle.

7. The intubation device of claim 1 wherein the power source consists of disposable batteries.

8. The intubation device of claim 1 wherein the power source consists of rechargeable batteries.

9. The intubation device of claim 1 wherein the distal tip of said stylet contains warming elements to defog the camera lens, the warming elements powered by the power source.

10. The intubation device of claim 1 wherein the flexible stylet portion is sealed without conduits.

11. The intubation device of claim 1 wherein the stylet is attached to the handle via a locking ball and socket type joint.

12. The intubation device of claim 1 further comprising at least one foil package stored with the intubation device, the at least one foil package containing in combination: a soft, lint-free, disposable, absorbent cleaning cloth; chemicals or chemical mixtures to clean, disinfect, lubricate, defog and warm the stylet and lens.

13. The intubation device of claim 1 further comprising a storage container to provide protection and rapid availability of the intubation device.
